(19) United States
(12) Patent Application Publication
(10) Pub. No.: US 2008/0033537 A1
(43) Pub. Date: Feb. 7, 2008

(54) BIODEGRADABLE STENT HAVING AN ACTIVE COATING

(75) Inventor: Michael Tittelbach, Nuernberg (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/834,432

(22) Filed: Aug. 6, 2007

(30) Foreign Application Priority Data
Aug. 7, 2006 (DE) 10 2006 038 236.6

(51) Int. Cl. A61F 2/82

(52) U.S. Cl. 623/1.42

(57) ABSTRACT

A stent having a main body made of a biodegradable material and an active coating applied to the main body, which comprises a biodegradable carrier matrix and at least one pharmaceutically active substance embedded in the carrier matrix.

### PRIORITY CLAIM

This patent application claims priority to German Patent Application No. 10 2006 038 236.6, filed Aug. 7, 2006, the disclosure of which is incorporated herein by reference in its entirety.

### FIELD

The present disclosure relates to a biodegradable stent having an active coating.

### BACKGROUND

For more than two decades, the implantation of endovascular support systems has been established in medical technology as one of the most effective therapeutic measures in the treatment of vascular illnesses. For example, in interventional treatment of stable and unstable angina pectoris, the insertion of stents has resulted in a significant reduction of the restenosis rate and thus to better long-term results. The main cause for the use of stent implantation in the event of the above-mentioned indication is the higher primary lumen obtained. An optimal vascular cross-section, which is primarily necessary for successful treatment, may be achieved by the use of a stent; however, the permanent presence of a foreign body of this type incites bodily processes which may result in gradual growing over of the stent lumen.

One approach for solving these problems is to manufacture the stent from a biodegradable material. Greatly varying materials are available to medical technicians for implementing biodegradable implants of this type. In addition to numerous polymers, which are frequently of natural origin or are at least based on natural compounds for better biocompatibility, more recently, metallic materials, having their more favorable mechanical properties, which are essential for implants, have been favored. Materials containing magnesium, iron, and tungsten have received special attention in this context.

A second approach for reducing the restenosis danger is the local application of pharmaceutical substances (active ingredients) which are intended to counteract the various mechanisms of pathological vascular changes at the cellular level and/or are intended to support the course of healing. The pharmaceutical substances are typically embedded in a carrier matrix in order to (i) influence an elution characteristic of the pharmaceutical substance, (ii) support adhesion of the coating on the implant surface, and (iii) optimize the production of the coating, in particular, the application of a defined quantity of active ingredients.

Materials of greatly varying embodiments have proven themselves as a carrier matrix. One may differentiate between permanent coatings and coatings made of a biodegradable carrier matrix. The coatings made of a biodegradable carrier matrix typically make use of polymers of biological origin. Carrier matrix, pharmaceutical substance, and possibly further auxiliary materials together implement a so-called “active coating” on the implant.

Combining the two above-mentioned approaches to reduce the restenosis rate further and support the healing process suggests itself. In particular, a combination of a biodegradable implant main body with an active coating which is also biodegradable may be advantageous.

It has been shown that the active coating has a significant influence on the degradation behavior of the implant main body; areas which are covered over a large area by the active coating are not accessible to the bodily medium, typically blood, and thus (locally) slow the degradation. As a result, fragmentation or, due to the correspondingly lengthened presence of the implant in the body, increase of the restenosis rate may occur. It is conceivable, in principle, to optimize the degradation behavior of the implant main body and active coating by variation of the material of carrier matrix and main body, the layer thickness of active coating, the design of the main body, and possibly the composition of the carrier matrix (content of pharmaceutically active substance, auxiliary materials) for a concrete implant; however, this is very complex and the results are not readily transferable to new developments without further measures.

A further problem is the influence of the process of degradation of the implant main body on the release of the pharmaceutically active substance from the carrier matrix. The degradation products of the main body may influence both the release of the substance from the carrier matrix and also the degradation of the carrier matrix, and thus, in turn, the release of the substance indirectly. In other words, the three processes of (i) release of the substance, (ii) degradation of the carrier matrix, and (iii) degradation of the implant main body interact and the local coincidence of the processes makes optimizing the implant more difficult.

### SUMMARY

The present disclosure provides an exemplary embodiment of the present invention, which is discussed below.

One aspect of the present disclosure provides a stent having a main body, comprising a biodegradable material, and an active coating applied to the main body, the coating comprising a biodegradable carrier matrix and at least one pharmaceutically active substance embedded in the carrier matrix, wherein the active coating has a degradation speed less than a degradation speed of the main body; and wherein the active coating is applied on a coating area of the surface of the main body provided for this purpose such that the coating area is divided into an uncoated partial area and a partial area coated with the active coating, the coated partial area covering 5 to 80% of the surface of the coating area; a distance of an arbitrary point of the surface in the coated partial area to the closest uncoated partial area is less than 35 μm; and a distance of an arbitrary first boundary point of the surface in the coated partial area to a second boundary point in the same coated partial area, which is furthest away from the first boundary point, is at most 400 μm.

The present disclosure is based in part on the finding that an application of the active coating in the coating area provided for this purpose which is delimited in area in the above-mentioned scope and an adaptation of the coating pattern while maintaining the predefined distance results in disentanglement of the degradation processes of carrier matrix and main body. In this way, it is possible to tailor the release of the pharmaceutically active substance and procedures during the degradation more precisely and possibly to restrict required modifications to only a part of the system. Because of the main framework degradation, the coated partial areas will detach from the surface of the main body and, if the coated partial areas are in contact with tissue, grow into the surrounding tissue. The coated partial areas function in the surrounding tissue as local active ingredient depots which are not in contact with the main framework of the implant either locally or in regard to the release and degradation processes.

In a preferred exemplary embodiment, the release speed of the pharmaceutically active substance is greater than the degradation speed of the carrier matrix, but less than the degradation speed of the main body. In this way, more precise setting of the dosing of the pharmaceutically active substance in the range limits established by the treatment plan may occur, because interfering interactions with the degradation processes of the implant main body and the carrier matrix are avoided or at least reduced. Preferably, the release speed is at least twice the degradation speed of the carrier substance, so that the quantity of substance which is released by diffusion processes from the carrier matrix, and not as a result of the decomposition of the carrier matrix, is increased. An advantage is that the substance released by diffusion is at least provided in a more adequate modification for resorption in the body. Moreover, because of a reduced interaction between the cited processes, a modification of the system, for example, to adapt to an individual treatment plan, is simplified.

The degradation speed of the main body is preferably 1.1 to 50 times the degradation speed of the active coating. At a degradation speed below the cited range limits, the danger of undesired interactions between the two degradation processes increases. At a degradation speed above the cited range limits, the dwell time of the active coating parts in the body is significantly lengthened, so that rejection reactions become more probable.

The coated partial area preferably covers 5 to 20% of the surface of the coating area. Above the cited limits, an attack area for the bodily medium is reduced so much that a noticeable delay of the main body degradation in the coating area occurs and thus an interaction of the cited processes may be reinforced.

The distance from an arbitrary point of the surface in the coated partial area to the closest uncoated partial area is preferably less than 30 μm. Above the cited limits, the danger exists that the coated partial area will delay the degradation of the main framework locally, namely, precisely where the distance to the boundary of the coated partial area is too large. As a result, artifacts may form and ingrowth of the active coating and its action as an active ingredient depot is obstructed.

The distance from an arbitrary first boundary point of the surface in the coated partial area to a second boundary point, which is furthest away from the first boundary point, is preferably at most 200 μm, more particularly at most 100 μm. Above the cited limits, the danger exists that the coated partial area will locally delay the degradation of the main framework. As a result, artifacts may form and ingrowth of the active coating and its action as an active ingredient depot may be obstructed.

Furthermore, the active coating preferably comprises multiple coating islands. These preferably have a mean diameter of 10 to 100 μm. The production process may be made especially simple by the contouring and the diameter delimitation, and modifications are more easily possible, e.g., for adapting the dosing of the active substance.

The uncoated partial area is preferably divided into multiple partial surfaces. Furthermore, partial surfaces having a size of up to 1000 μm² preferably occupy at least 70% of the total surface of the uncoated partial area. In this way, it is ensured that an attack surface for a bodily medium in the uncoated partial area is sufficiently large so that wetting with the active medium is easier. Otherwise, a significant delay of the main body degradation in the coating area may occur.

The main framework of the stent is preferably molded from a magnesium, iron, or tungsten out. Magnesium alloys of the type WE, in particular, WE43 are especially preferred. WE43 is distinguished by the presence of rare earth elements and yttrium. The cited materials may be processed easily, have low material costs, and are especially suitable for vascular supports because of the relatively rapid degradation and the more favorable elastic behavior than polymers (lower recoil of the stent). Furthermore, a positive physiological effect of the degradation products on the healing process has been established for at least a part of the alloys. Moreover, it has been shown that magnesium stents produced from WE43 do not generate any interfering magnetic resonance artifacts, as are known, for example, from medical stainless steel (316A), and, therefore, treatment success may be tracked using detection devices based on magnetic resonance. The biodegradable metal alloys made of the elements magnesium, iron, or tungsten preferably contain the cited elements in a proportion of at least 50 weight-percent, in particular at least 70 weight-percent, especially preferably at least 90 weight-percent of the alloy.

### BRIEF DESCRIPTION OF THE DRAWINGS

The present disclosure is explained in the following on the basis of an exemplary embodiment and the attached drawings.

FIG. 1 shows a schematic top view of a detail of a biodegradable implant having a coating according to the present disclosure; and

FIG. 2 shows a section through the main body of the stent in area of active coating.

### DETAILED DESCRIPTION

For purposes of the present disclosure, the term “biodegradable” relates to a material which is degraded in vivo, i.e., loses its mechanical integrity. The degradation products do not necessarily have to be completely resorbed or excreted by the body. For example, small particles may also remain at the location of application. For purposes of the present disclosure, biodegradation relates, in particular, to hydrolytic, enzymatic, and other degradation processes in the living organism caused by the metabolism, which result in gradual dissolving of at least large parts of the materials used. The term biocorrosion is frequently used synonymously with biodegradation. For purposes of the present disclosure, the term bioresorption additionally comprises the subsequent resorption of the degradation products.

For purposes of the present disclosure, an “active coating” comprises a biodegradable carrier matrix and at least one pharmaceutically active substance embedded therein. Optionally, the active coating may also contain further auxiliary materials to improve adhesion capability and processability and the release of the substance, for example. In addition, polymers of natural origin come into consideration as materials for the carrier matrix, such as hyaluronic acid, poly-L-lactide, poly-D-lactide, collagen, and the like.

The carrier matrix used is preferably based on a biodegradable polymer. Biodegradable polymers have been known for some time and are also used for oral applications and injections. Many different polymer classes have been used for medical purposes, each of which have properties custom tailored for the corresponding use. The polymer system used must be examined in relation to the physiological effect; the degradation products may not be toxic and/or form toxic substances by reaction with bodily substances. Furthermore, it is to be ensured that a potential of the polymer systems for initiating infections because of foreign body reactions of the immune system is as low as possible. Finally, an interaction between the active ingredient and the polymer matrix must be taken into consideration; the polymers may neither lose their biodegradable properties by interaction with the active ingredient nor may the active ingredient be deactivated by reaction of the active ingredient with the polymer matrix. Therefore, one skilled in the art will take the cited parameters into consideration when selecting a specific system made of polymer matrix and active ingredient.

For purposes of the present disclosure, a “pharmaceutically active substance” includes, but is not limited to, a vegetable, animal, or synthetic active ingredient which is used at suitable dosing as a therapeutic agent for influencing states or functions of the body, as a replacement for natural active ingredients generated by the human or animal body, and for removing or making harmless pathogens or bodily foreign materials. The release of the substance in the implant surroundings has a positive effect on the course of healing and/or counteracts pathological changes of the tissue as a result of the surgical intervention.

For purposes of the present disclosure, the “release of pharmaceutically active substance” is the removal of the substance from the carrier matrix. A partial process for the release of pharmaceutically active substance is the dissolving of absorbed substances out of the solid or gel-type carrier matrix with the aid of media present in the body, such as blood.

A release speed is determined as follows: a half-life is detected, in which 50 weight-percent of the substances released, and a (mean) release speed is determined on the basis of the half-life for assumed linear release kinetics.

A degradation speed of the carrier matrix and the main body is detected in that, first a half-life is ascertained, in which 50 weight-percent of the material forming the main body and/or the carrier matrix is degraded, and then a (mean) speed of the degradation processes calculated on the basis of this half-life for an assumed linear course of the degradation.

The main framework of the stent comprises all components necessary for ensuring the mechanical integrity and main functionalities of the implant. In addition, the stent may have marker elements, for example, which are bonded to the main body in a suitable way. The main framework provides a surface which is used for applying the active coating. An area of the coating may be established individually; preferably, only an outwardly directed part of the main framework is coated.

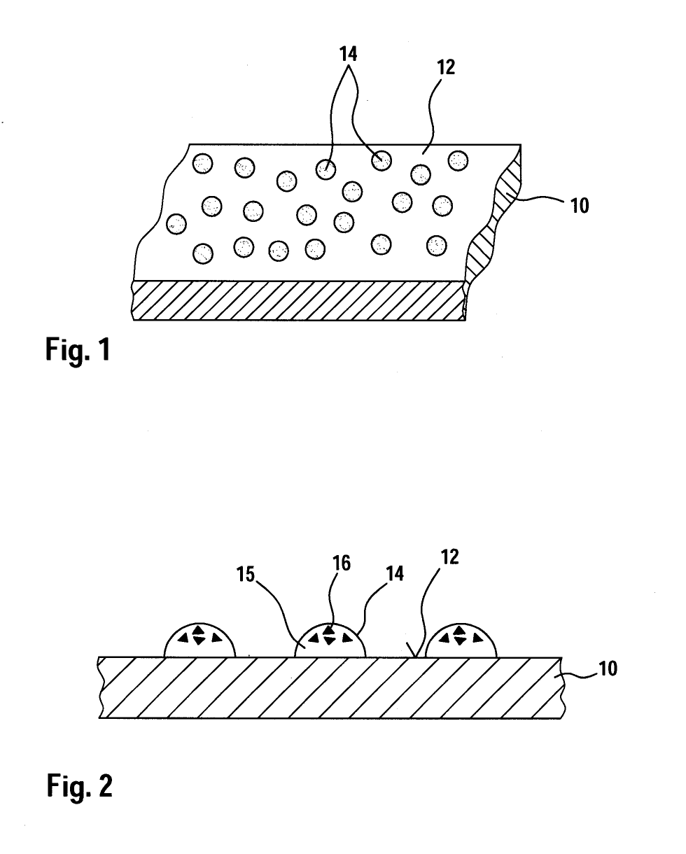

FIG. 1 shows a section of the main body 10 of the stent which is molded from a biodegradable material. The metallic material forms a filigree framework of struts connected to one another, whose design is only of subordinate significance for the present disclosure. An active coating is applied to an external surface 12 of the main body 10. As is obvious, the coating area is divided into an uncoated partial area and a partial area coated with the active coating.

The active coating is implemented as multiple coating islands 14 which comprise a biodegradable carrier matrix 15 and at least one pharmaceutically active substance 16 (shown here as a triangle) embedded in the carrier matrix 15. The coating islands 14 are applied to the surface 12 of the main body 10 in such a way that the coated partial area, i.e., the coating islands 14, cover approximately 10-15% of the surface 12 of the coating area.

The main body 10 comprises the magnesium alloy WE43, and the carrier matrix is high-molecular-weight poly-L-lactide (molar mass greater than 500 kD). A degradation speed of the polymer material of the carrier matrix 15 is approximately 10 to 15 times the degradation speed of the material of the main body 10.

The individual coating islands have a mean diameter of approximately 50 to 70 μm. A distance of an arbitrary point of the surface in the coated partial area to the closest uncoated partial area is thus less than 35 μm. If the coating islands are uniformly round, the distance from an arbitrary first boundary point of the surface of the coated partial area to a second boundary point, which is furthest away from the first boundary point, is approximately 50 to 70 μm.

The following procedure may be used for applying the coating islands 14.

The stent is pre-mounted on a balloon or catheter. A solution or extremely fine dispersion of the biodegradable polymer and the at least one active substance is provided in a reservoir. Subsequently, droplets of defined size are applied in selected areas of the main body via a controllable microinjection system. The solvent is withdrawn by vaporization and the coating islands of defined diameter are formed.

What is claimed is:

1. A stent having a main body, comprising:
(a) a biodegradable material, and
(b) an active coating applied to the main body, the coating comprising a biodegradable carrier matrix and at least one pharmaceutically active substance embedded in the carrier matrix,
wherein the active coating has a degradation speed less than a degradation speed of the main body; and wherein the active coating is applied on a coating area of the surface of the main body provided for this purpose such that the coating area is divided into an uncoated partial area and a partial area coated with the active coating, the coated partial area covering 5% to 80% of the surface of the coating area; a distance of an arbitrary point of the surface in the coated partial area to the closest uncoated partial area is less than 35 μm; and a distance of an arbitrary first boundary point of the surface in the coated partial area to a second boundary point in the same coated partial area, which is furthest away from the first boundary point, is at most 400 μm.

2. The stent of claim 1, wherein the degradation speed of the main body is 1.1 to 50 times the degradation speed of the active coating.

3. The stent of claim 1, wherein the coated partial area covers 5% to 20% of the surface of the coating area.

4. The stent of claim 1, wherein the distance of an arbitrary point of the surface of the coated partial area to the closest uncoated partial area is less than 30 μm.

5. The stent of claim 1, wherein a release speed of the pharmaceutically active substance is greater than the degradation speed of the carrier matrix, but less than the degradation speed of the main body.

6. The stent of claim 1, wherein the active coating comprises multiple coating islands.

7. The stent of claim 6, wherein the coating islands have a mean diameter of 10 to 100 μm.

8. The stent of claim 1, wherein the uncoated partial area is divided into multiple partial surfaces, and partial surfaces having a size of up to 1000 μm² occupy at least 70% of the total surface of the uncoated partial area.
